(19) United States
(12) Patent Application Publication
(10) Pub. No.: US 2011/0123630 A1
(43) Pub. Date: May 26, 2011

(54) TOPICAL TREATMENT OF PERIPHERAL NEUROPATHY

(76) Inventors: Sam Schwartz, Woodland Hills, CA (US); Danny Petrasek, Los Angeles, CA (US)

(21) Appl. No.: 13/015,314

(22) Filed: Jan. 27, 2011

Related U.S. Application Data
(63) Continuation of application No. 12/110,131, filed on Apr. 25, 2008, now abandoned.
(60) Provisional application No. 60/926,294, filed on Apr. 25, 2007.

(51) Int. Cl. A61K 9/50; A61K 31/385; A61K 36/886; A61K 31/51; A61P 29/00

(52) U.S. Cl. 424/490; 514/440; 424/744; 514/276

(57) ABSTRACT

A method and composition for the treatment of diabetic neuropathy is disclosed. A preferred embodiment of the invention relates to a topical composition for the treatment of systems related to diabetic neuropathy by application to the affected area. The composition provides relief of these symptoms and inhibits those factors that contribute to these systems, such as an aldose reductase inhibitor. A preferred embodiment of the invention relates to a method of treatment for diabetic neuropathy utilizing a regime of administrations of a topical composition in amounts effective to counter those areas of the body affected by peripheral Neuropathy. Yet another preferred embodiment of the invention relates to a method of improvement of those areas effected by diabetic peripheral neuropathy using a topical composition over an effective period of time.

### CROSS REFERENCE TO RELATED APPLICATIONS

This patent application is a continuation of copending U.S. patent application No. 12/110,131, filed Apr. 25, 2008, entitled TOPICAL TREATMENT OF PERIPHERAL NEUROPATHY, which claims priority to U.S. Provisional Patent Application No. 60/926,294, filed Apr. 25, 2007, entitled COMPOSITION AND METHOD FOR TREATING DIABETIC PERIPHERAL NEUROPATHY, the disclosures of which are incorporated herein by reference in their entirety.

FIELD

The present invention relates to a method and composition for the topical treatment of diabetic neuropathy. The present invention further relates to a topical composition including effective antioxidants for temporary relief of symptoms of peripheral neuropathy associated with diabetes and to a method for administering the topical composition.

### BACKGROUND

Peripheral neuropathy is estimated to eventually afflict roughly 30-70 percent of persons suffering from diabetes mellitus, a common disease that can be classified into insulin dependent and non-insulin-dependent types. The former is usually treated by a strict diet along with insulin, the latter with drugs.

Peripheral neuropathy describes damage to the peripheral nervous system, the vast communications network that transmits information from the central nervous system (brain and spinal cord), throughout the entire body. Peripheral nerves also send sensory information back to the central nervous system, such as a message that the body's extremities are suffering from cold or heat, or that nasal passages are irritated. Damage to the peripheral nervous system interferes with these vital connections by distorting or interrupting communications between the brain and the rest of the body.

As consequence, various problems and a wide spectrum of symptoms stemming from this kind of nerve damage can manifest within the body in that every peripheral nerve has a highly specialized function in a specific part of the body. Some persons afflicted with this malady experience sensing issues like temporary numbness, a tingling and burning sensation in their hands and feet. Others may suffer more extreme symptoms, including muscle wasting, paralysis, or organ or gland dysfunction, or organ failure.

More than 100 types of peripheral neuropathy have been identified, each with its own characteristic set of symptoms, pattern of development, and prognosis. Impaired function and symptoms depend on the type of nerves—motor, sensory, or autonomic—that are damaged. Motor nerves control movements of all muscles under conscious control, such as those used for walking, grasping things, or talking. Sensory nerves transmit information about sensory experiences, such as the feeling of a light touch or the pain resulting from a cut, Autonomic nerves regulate biological activities that people do not control consciously, such as breathing, digesting food, and heart and gland functions. Although some neuropathies may affect all three types of nerves, others primarily affect one or two types. Therefore, doctors may use terms such as predominately motor neuropathy, predominately sensory neuropathy, sensory-motor neuropathy, or autonomic neuropathy to describe a patient's condition.

Diabetic neuropathy is currently categorized into three groups comprising mononeuropathy, symmetrical peripheral polyneuropathy and autonomic neuropathy (Williams Text Book of Endocrinology, 8th Edition, p. 1301, Harrison's Principles of Internal Medicine, 12th Edition, p. 1754. Although the exact cause of peripheral neuropathy is not known, what is known is that the disease may be either inherited or acquired. The National Institute of Neurological Disorders and Stroke has identified various potential causes of peripheral neuropathy. See http://www.rinds.nih,gpy/disorders/eri heralneuropath.

As to acquired peripheral neuropathy, the causes include physical injury or trauma to a nerve, tumors, toxins, autoimmune responses, nutritional deficiencies, alcoholism, and vascular and metabolic disorders.

Kidney disorders can lead to abnormally high amounts of toxic substances in the blood that can severely damage nerve tissue. A majority of patients who require dialysis because of kidney failure develop polyneuropathy: Some liver diseases also lead to neuropathies as a result of chemical imbalances.

Hormonal imbalances can disturb normal metabolic processes and cause neuropathies. For example, ate underproduction of thyroid hormones slows metabolism, leading to fluid retention and swollen tissues that can exert pressure on peripheral nerves, Overproduction of growth hormone can lead to acromegaly, a condition characterized by the abnormal enlargement of many parts of the skeleton, including the joints. Nerves running through these affected joints often become entrapped.

Vitamin deficiencies and alcoholism can cause widespread damage to nerve tissue. Vitamins E, B1, B6, B12, and niacin are essential to healthy nerve function. Thiamine deficiency, in particular, is common among people with alcoholism because they often also have poor dietary habits, Thiamine deficiency can cause a painful neuropathy of the extremities. Some researchers believe that excessive alcohol consumption may, in itself, contribute directly to nerve damage, a condition referred to as alcoholic neuropathy.

Vascular damage and blood diseases can decrease oxygen supply to the peripheral nerves and quickly lead to serious damage to or death of nerve tissues, much as a sudden lack of oxygen to the brain can cause a stroke. Diabetes frequently leads to blood vessel constriction. Various forms of vasculitis (blood vessel inflammation) frequently cause vessel walls to harden, thicken, and develop scar tissue, decreasing their diameter and impeding blood flow. This category of nerve damage, in which isolated nerves in different areas are damaged, is called mononeuropathy multiplex or multifocal mononeuropathy.

Connective tissue disorders and chronic inflammation can cause direct and indirect nerve damage. When the multiple layers of protective tissue surrounding nerves become inflamed, the inflammation can spread directly into nerve fibers. Chronic inflammation also leads to the progressive destruction of connective tissue, making nerve fibers more vulnerable to compression injuries and infections, Joints can become inflamed and swollen and entrap nerves, causing pain.

Repetitive stress frequently leads to entrapment neuropathies, a special category of compression injury. Cumulative damage can result from repetitive, forceful, awkward activities that require flexing of any group of joints for prolonged periods. The resulting irritation may cause ligaments, tendons, and muscles to become inflamed and swollen, constricting the narrow passageways through which some nerves pass. These injuries become more frequent during pregnancy, probably because weight gain and fluid retention also constrict nerve passageways.

Toxins can also cause peripheral nerve damage. People who are exposed to heavy metals (arsenic, lead, mercury, thallium), industrial drugs, or environmental toxins frequently develop neuropathy. Certain anticancer drugs, anticonvulsants, antiviral agents, and antibiotics have side effects that can include peripheral nerve damage, thus limiting their long-term use. Also, infections and autoimmune disorders can be a cause.

As to inherited forms of peripheral neuropathy, they may be caused by inborn mistakes in the genetic code or by new genetic mutations. Some genetic errors lead to mild neuropathies with symptoms that begin in early adulthood and result in little, if any, significant impairment. More severe hereditary neuropathies often appear in infancy or childhood.

There are many approaches to reducing or preventing these forms of damage, which are collectively known as the long-term complications of diabetes. Drugs and diet are not enough to prevent peripheral neuropathy.

Among various neurologic tests, nerve conduction velocity (NCV) examination is the most widely used as a method of objectively evaluating severity of diabetic, neuropathy.

Using two locations for motor nerves, both are selected to be stimulated with intensities selected to induce the largest peak for each of the corresponding controlling muscles in electromyograms. The distance between the two locations is then divided by the balance between the obtained latencies in the two electromyograms. The sensory nerve is electrically stimulated in the orthodrornic direction at an intensity selected to obtain the largest action potential, and then the distance between the stimulated location and the induced location is divided by the latency.

One reported approach concerned some trial treatments have been conducted during the 1970s and 1980 based upon the hypothesis that abnormality of metabolic factors is viewed as a cause, Greene D A, De Jesus P V Jr., et al. (Effects of insulin and dietary myoinositoi on impaired peripheral motor nerve conduction velocity in acute streptozatocin diabetes, J. Clin. Invest, 1975, 55, 6, 1326-3.6); Yagihashi Nishihira M., et al. (Morphometrical analysis of the peripheral nerve lesions in experimental diabetes rats, Tohoku J. Exp. Med., 129, 2, 139-49, 1979). These studies confirmed that peripheral nerve fibers of model rats for diabetic neuropathy were morphologically impaired and NMI was reduced; and, it was reported that when insulin was administered to the rats, improvements in NCV could be observed, thus finding that control over blood glucose level led to improvements in NCV.

The glucose control approach is aimed at the over-production of the glucose metabolite, sorbitol, in the cells of the body. High levels of sorbitol may be among the causes of diabetic neuropathy. Pharmaceutical companies have been developing aldose reductase inhibitors for the purpose of reducing diabetic neuropathy.

Also, a wide variety of flavanoids are effective inhibitors of aldose reductase, including such flavanoids as quereetin, quereetin and myrecetrin. However, studies have shown that a need exists for aldose reductase inhibitors that can be more effectively used and in lower doses than the prior art compounds, including these flavanoids. Scientific efforts have concentrated on improving aldose reductase inhibitors.

Another approach to the treatment of neuropathy is using pharmaceutical compositions for stimulating the growth of neurites in nerve cells comprising a neurotrophic amount of a compound and a nerve growth factor. These compositions may be administered in a number of ways including orally and topically.

Yet another approach to the treatment of neuropathy is where compositions suitable for treatment of vitamin H deficiencies are administered for the treatment of neuropathy. One such composition comprises biotin salts with alkanolamines, which may be administered orally, parenterally, or topically.

Still yet another approach relates to the treatment of diabetic peripheral neuropathies by periodic topical application of a composition containing capsicum oleoresin as the active ingredient. When applied to the skin of the affected area, pain and burning associated with the neuropathy are said to be reduced. However, capsicum oleoresin has been shown to kill nerve endings in some cases and thus this composition suffers from this disadvantage.

There is also a method of treatment of diabetic neuropathy using combined administration of a formulation including as an active ingredient, a prostaglandin I derivative with an anti-diabetic agent in order to improve nerve conduction velocities. Suitable anti diabetic agents include oral hypoglycemic agents and insulin.

Current treatment methods; include dietetic therapy, administration of insulin, administration of aldose reductase inhibitors or arninoguaninidine to improve abnormal glucose metabolism, administration of troglitazone or agents for the improvement of blood flow have been tested but found to be insufficient when a single drug was used. Studies have disclosed that methods of treatment by combined use of different therapeutic agents which have different functions had yet to be established.

No adequate results, however, have been practically provided by the treatments for diabetic neuropathy targeting improvements t f NCV. In detail, Pietri A, et al. (Changes in nerve conduction velocity after six weeks of glucoregulation with portable insulin infusion pumps. Diabetes, 29, 8, 668, 1980); Graf R J, et al. (Glycemic control and nerve conduction abnormalities in non-insulin-dependent diabetic subjects, Ann. Intern. Med., 94, 3, 307, 1981) separately gave drug therapies using insulin to patients with insulin-dependent diabetes and patients with non-insulin-dependent diabetes in 1980 and 1981, respectively, in order to treat diabetic neuropathy. It was consequently reported that motor nerve conduction velocity (MCV) was improved by controlling blood glucose level. It was also reported, however, that only by controlling blood glucose level, improvements in sensory nerve conduction velocity (SCV) could not be observed, and that treatments mainly proposing to control blood glucose level, accordingly, only provided very limited improvements of functions such as thermal sensitivity and vibratory sensibility.

As can be seen, the need exists in the art for an effective treatment for diabetic neuropathy without severe side effects, as do many of the methods above, as do many arose reductase inhibitors.

Accordingly, an object of a preferred embodiment of the present invention is to provide a topical composition that is effective for the treatment of diabetic neuropathy.

Another object of a preferred embodiment of the present invention is to provide a method of use of the topical composition in treating symptoms of diabetic neuropathy.

Another object of a preferred embodiment of the present invention is to provide a topical composition for the treatment of diabetic neuropathy which does not cause severe side effects in the patients treated with the composition.

These and other objects of the present invention will be apparent from the summary and detailed descriptions of the invention which follow.

### SUMMARY

The foregoing needs are met, to a great extent, by the present disclosure, wherein, in one respect, a topical agent and method is provided that in some embodiments is used to treat peripheral neuropathy.

The preferred embodiment of the present invention relates to a topical composition for the treatment of systems related to diabetic neuropathy by application to the affected area. The composition provides relief of these symptoms and inhibits those factors that contribute to these systems, such as an aldose reductase inhibitor.

Another preferred embodiment of the present invention relates to a method of treatment for diabetic neuropathy utilizing a regime of administrations of a topical composition in amounts effective to counter those areas of the body affected by peripheral neuropathy.

Another preferred embodiment of the present invention relates to a method of improvement of those areas effected by diabetic peripheral neuropathy using a topical composition over an effective period of time.

There has thus been outlined, rather broadly, certain embodiments that the detailed description thereof herein may be better understood, and in order that the present contribution to the art may be better appreciated. There are, of course, additional embodiments that will be described below and which will form the subject matter of the claims appended hereto.

In this respect, before explaining at least one embodiment in detail, it is to be understood that embodiments are not limited in its application to the details of construction and to the arrangements of the components set forth in the following description or illustrated in the drawings. In addition to the embodiments described, the various embodiments are capable of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the disclosure. It is important, therefore, that the claims be regarded as including such equivalent constructions insofar as they do not depart from the spirit and scope of the various embodiments.

### DETAILED DESCRIPTION

In general, the composition includes one or more of, alpha lipoic acid, Dead Sea mineral salt, grape seed extract; thiamin, selenium, aloe vera. The alpha lipoic acid can be encapsulated in micro capsules, and then added to a base cream that contains glycerin, sunflower oil, octyl palmitate, caprylic capric triglyceride, glyceryl sterarate, peg-100 stearate, eugenol, oat extract, chamomile, calendula, green tea extract, stearic acid, cetearyl alcohol, ceteareth-20, tocopheryl acetate, dimethicone, xanthan gum, methyl-paraben, dmdm hydantoiin, and fragrance. In a particular example, the composition includes:

| Phase | Parts | Ingredient |
|---|---|---|
| 1 | 61.9500 | Deionized Water |
| | 1.0000 | Aloe Vera Juice |
| | .2000 | Methyparaben |
| | 3.0000 | Propylene Glycol |
| | .1000 | Keltrolt |
| 3 | 1.0000 | Sunflower Oil |
| | 2.0000 | Kessco OP |
| | 2.0000 | Liponate GC |
| | 1.0000 | Thodosil |
| | 2.0000 | Hydrogenated Vegetable Oil |
| | 3.5000 | Lipo GMS-Witcnol 2407 |
| | 2.0000 | Stearic Acid |
| | 2.0000 | Lipomulse 165/Arlacel |
| | 1.0000 | Lipocol C/Alfol |
| | 1.0000 | Lipowax D |
| | 0.1000 | Propylparaben |
| | 0.1000 | Vitamine E Acetate |
| 4 | 2.0000 | Eugenol Extra USP |
| | .04000 | Mackstat DM |
| | 0.0500 | White Diamond B/M |
| | 0.1000 | Coviox |
| 5 | 7.8600 | Deionized Water |
| | 5.0000 | Dead Sea Salt |
| | 1.0000 | Alpha Lipoic Acid |
| | 100.0000 | |

In addition to the ingredients listed above, one or more of Thiamine, grape seed extract, and flax seed extract may be added at a concentration of less than 1% each.

The topical composition contains an alpha lipoic acid (ALA) in the form of the active isomer of Lipoic Acid “R lipoic acid.” In various forms, the ALA may be encapsulated in any suitable manner. In a particular example of a manner of encapsulation suitable for use with an embodiment of the invention, the ALA may be encapsulated in a mixture of gelatin, corn oil, and water. More specifically, the ALA may be encapsulated in micro-droplets having a size of about 70 microns. In general, the gelatin coating material may makeup approximately 7.1% by weight of the encapsulated ALA and the core ALA material may be approximately 92.9% by weight.

As mentioned, the topical contains two independent ingredients known to help in neuropathy: Thiamine and Selenium. Both which operate with independent mechanisms and would be synergistic and not a simple addition or linear combination to the ALA.

The amount of ALA topically used here is in a much higher effective dose than would be available from oral tablets because the oral tablets have a large volume of distribution.

The micro-encapsulation ensures deeper and more efficient and longer penetration and stability.

In addition, the use of anti-microbial Dead Sea salts provide another barrier of defense against associated complications of neuropathy. Microbes potentially create skin lesions that get infected and create ulcers which promote further neuropathic degeneration.

To test the efficacy of the topical treatment, a double blind prospective study may be conducted with both positive and negative controls. This study may be utilized to evaluate the effects of applying ALA topically on patients exhibiting symptoms of peripheral neuropathy. In general, the peripheral neuropathy may be caused by a variety of disease states and/or as a result of various treatments, such as, chemotherapy induced neuropathy. In a particular example, the peripheral neuropathy may diabetes related. The size of the study may, initially include 20-50 patients and the study may have a duration of approximately 3 months.

It is an advantage of various embodiments that, by administering ALA topically, a grater concentration of ALA may be delivered in and around affected neurons as compared to oral administration of ALA.

The foregoing detailed description of the invention and examples are not intended to limit the scope of the invention in any way and should not be construed as limiting the scope of the invention.

The many features and advantages of the various embodiments are apparent from the detailed specification, and thus, it is intended by the appended claims to cover all such features and advantages that fall within the true spirit and scope of the embodiments. Further, since numerous modifications and variations will readily occur to those skilled in the art, it is not desired to limit the embodiments to the exact construction and operation illustrated and described, and accordingly, all suitable modifications and equivalents may be resorted to, falling within the scope of the various embodiments.

What is claimed is:

1. A composition for topical application for the relief of pain due to peripheral neuropathy, the composition comprising:
alpha lipoic acid in a microencapsulated form, Dead Sea salts, and eugenol, all of the foregoing in a pharmaceutically acceptable dosage and in a topical ointment suitable for application to the skin, wherein the composition is useful for providing pain relief to areas below the skin.

2. The composition of claim 9, further comprising:
thiamine.

3. The composition of claim 9, further comprising:
deionized water, 69.81 parts;
aloe vera juice, 1 part;
methyparaben, 0.2 parts;
propylene glycol, 3 parts;
xanthan gum, 0.1 part;
sunflower oil, 1 part;
hexadecanoic acid,2-ethylhexyl ester, 2 parts;
caprylic/capric triglyceride, 2 parts;
thodosil, 1 part;
hydrogenated vegetable oil, 2 parts;
glyceryl stearate, 3.5 parts;
stearic acid, 2 parts;
glyceryl stearate and PEG-100 stearate/Arlacel, 2 parts;
PEG-40 hydrogenated castor oil /Alfol, 1 part;
cetearyl alcohol and ceteareth-20, 1 part;
propylparaben, 0.1 part;
vitamin E acetate, 0.1 part;
eugenol extra USP, 2 parts;
DMDM hydantoin, 0.04 parts;
White Diamond B/M, 0.05 parts;
vitamin E, 0.1 parts;
Dead Sea salt, 5 parts; and
alpha lipoic acid, 1 part.

4. The composition of claim 9, wherein the alpha lipoic acid is encapsulated in micro-droplets having a size of about 80 microns.
